(19) United States
(12) Patent Application Publication
(10) Pub. No.: US 2009/0264355 A1
(43) Pub. Date: Oct. 22, 2009

(54) METHODS TO TREAT ALZHEIMER'S DISEASE OR OTHER DISORDERS MEDIATED BY AMYLOID-BETA ACCUMULATION IN A SUBJECT

(75) Inventors: David Holtzman, St. Louis, MO (US); David Piwnica-Worms, Ladue, MO (US); John R. Cirrito, St. Louis, MO (US); Anne Fagan Niven, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 12/089,791

(22) PCT Filed: Oct. 11, 2006
(86) PCT No.: PCT/US06/39766

§ 371 Date: Sep. 8, 2008

Related U.S. Application Data
(60) Provisional application No. 60/726,292, filed on Oct. 13, 2005.

(51) Int. Cl. A61K 38/22; A61P 25/28

(52) U.S. Cl. 514/12

(57) ABSTRACT

The invention provides methods of delaying the onset, slowing the progression, preventing, or treating Alzheimers disease in a subject. In particular, the invention provides methods of modulating transport of Aβ across the blood brain barrier of the subject.

### CROSS REFERENCE TO RELATED APPLICATIONS

This application claims the priority of PCT Application PCT/US06/039766, filed Oct. 11, 2006, which claims the priority of U.S. provisional application No. 60/726,292, filed Oct. 13, 2005, each of which is hereby incorporated by reference in their entirety.

### GOVERNMENTAL RIGHTS

This invention was made with government support under AG13956, AG023316, and P50CA94056 awarded by the NIH. The government has certain rights in the invention.

### FIELD OF THE INVENTION

The invention relates to methods for delaying the onset, slowing the progression, preventing or treating Alzheimer's disease in a subject. In particular, the invention relates to modulating the concentration of Aβ in the brain interstitial fluid of a subject.

### BACKGROUND OF THE INVENTION

Alzheimer's Disease (AD) is the most common cause of dementia and is an increasing public health problem. It is currently estimated to afflict 5 million people in the United States, with an expected increase to 13 million by the year 2050. Alzheimer's Disease leads to loss of memory, cognitive function, and ultimately loss of independence. It takes a heavy personal and financial toll on the patient and the family. Because of the severity and increasing prevalence of the disease in the population, it is urgent that better treatments be developed.

Biochemical, genetic, and animal model evidence implicates amyloid-β (Aβ)) as a pathogenic peptide in AD. The neuropathologic and neurochemical hallmarks of AD include synaptic loss and selective neuronal death, a decrease in certain neurotransmitters, and the presence of abnormal proteinaceous deposits within neurons (neurofibrillary tangles) and in the extracellular space (cerebrovascular, diffuse, and neuritic plaques). The main constituent of plaques is Aβ, a 38-43 amino acid peptide cleaved from the amyloid precursor protein (APP). Throughout life, soluble Aβ is secreted primarily by neurons, but also other cell types. Multiple lines of evidence suggest that Aβ accumulation and change of conformation to forms with a high β-sheet structure is central in AD pathogenesis. In late-onset AD, the total amount of Aβ that accumulates in the brain is about 100-200-fold higher in AD brain homogenates versus control brains. Accumulation of Aβ first occurs in specific regions of the neocortex, including parts of the frontal, temporal, and parietal lobes, and the hippocampus; areas that have the earliest and most severe neuropathology in AD. Damage to these areas manifests in the first clinical symptoms of AD—memory and cognitive loss.

Aβ accumulation is dependent, in part, on the concentration of Aβ and the transport of Aβ out of cells. Because Aβ accumulation plays a central role in AD pathogenesis, there is a need in the art for methods of modulating Aβ accumulation, including modulating the concentration of Aβ and modulating the transport of Aβ.

### SUMMARY OF THE INVENTION

One aspect of the present invention is a method of delaying the onset, slowing the progression, preventing or treating Alzheimer's disease in a subject. Typically, the method comprises modulating P-glycoprotein mediated transport of Aβ across the blood brain barrier of the subject.

Another aspect of the present invention encompasses a method of modulating the concentration of Aβ in the brain interstitial fluid of a subject. Generally speaking, the method comprises modulating the P-glycoprotein mediated transport of Aβ across the blood brain barrier.

A further aspect of the present invention is a method to modulate the transport of Aβ from the brain interstitial fluid across the blood brain barrier in a subject. The method comprises modulating the activity of P-glycoprotein.

Other aspects and features will be in part apparent and in part pointed out hereinafter.

### BRIEF DESCRIPTION OF THE FIGURES

FIG. 1 depicts clearance of [¹²⁵I]Aβ₄₀ and [¹²⁵I]Aβ₄₂ from the brain after intracerebral microinjection. Two-to-three month old FVB wild type (Pgp-wild type) and mdr1a/b−/− (Pgp-null) mice on an FVB background were injected with 12 nmoles [¹²⁵I]Aβ₄₀ or [¹²⁵I]Aβ₄₂ into the striatum. Clearance of each peptide was assessed 30 minutes after treatment. [¹⁴C]inulin, an inert reference molecule, was co-injected in each experiment to assess bulk flow elimination of interstitial fluid (ISF). (A) Brain recovery (N_{b}/Nᵢ) of [¹²⁵I]Aβ₄₀ (left) and [¹⁴C]inulin (right). Significantly more [¹²⁵I]Aβ₄₀ was recovered in the brains of Pgp-null than Pgp-wild type mice (*p=0.0123). Similar levels of injected [¹⁴C]inulin were recovered in both groups. (B) Calculation of [¹²⁵I]Aβ₄₀ clearance via the blood-brain barrier (BBB) and ISF bulk flow mechanisms (from data in A). Significantly less Aβ₄₀ was cleared via the BBB in Pgp-null mice compared to Pgp-wild type mice (**p=0.0033). (C) Brain recovery of [¹²⁵I]Aβ₄₂ (left) and [¹⁴C]inulin (right). More [¹²⁵I]Aβ₄₂ was cleared from the brain in Pgp-wild type than in Pgp-null mice (^{#}p=0.0047). Approximately 85% of [¹⁴C]inulin was retained in both groups. (D) Similar to calculations for Aβ₄₀ clearance, significantly less Aβ₄₂ was cleared from the brain of Pgp-null mice compared to Pgp-wild type (^{##}p=0.0004). (E) LRP1 expression was decreased by 51% in cerebral vessels of 2-3 month old Pgp-null mice compared to Pgp wild type controls (⁺p=0.002). Relative absorbance representing the signal obtained from the 85 kDa LRP1 band on the Western blots normalized to the loading control is shown on the y-axis. Insert: representative lanes from an LRP1, 85 kD peptide (LRP1-85) Western blot and protein loading control. Values are mean±SEM, n=4-8 per group.

FIG. 2 depicts increasing Aβ levels in brain interstitial fluid (ISF) after acute pharmacologic inhibition of Pgp. Using in vivo microdialysis, we assessed the concentration of ISF Aβ₁₋ₓ within the hippocampus of 3 month old APPsw mice treated with XR9576, a Pgp inhibitor. For each animal, basal concentration of ISF Aβ₁₋ₓ was established over 5 hours, followed by intravenous injection of XR9576 and continued assessment of ISF Aβ for an additional 10 hours. Samples were collected at hourly intervals and assessed for Aβ₁₋ₓ using a sandwich ELISA. (A) Eight hours after intravenous administration of 80 mg/kg XR9576, ISF exchangeable Aβ₁₋ₓ (eAβ₁₋ₓ) levels began to increase and reached 127.7±3.4% of control-treated (vehicle-treated) mice (*p<0.05, n=5 per group). (B) Ten hours after treatment with either XR9576 or vehicle, cerebral capillaries were isolated and analyzed for levels of LRP1 expression by Western blot (n=5 per group). Levels of LRP1 in cerebral vessels did not change with acute inhibition of Pgp.

FIG. 3 depicts the bio-distribution of a Pgp substrate in APPsw, Pgp-null mice. Following tail-vein bolus injection of 2 μCi [^{99m}Tc]Sestamibi, brain content of [^{99m}Tc]Sestamibi in 2- to 4-month old wild type and Pgp-null mice (on an FVB background), as well as APPsw mice expressing or lacking Pgp was determined 5 min post-injection. More tracer was retained in all Pgp-null mice (*p=0.0043, FVB strain and **p=0.0058, APPsw^{+/-} strain, respectively), demonstrating, as expected, that Pgp normally hinders the entry of [^{99m}Tc]Sestamibi into the brain. Taken together, these findings suggest that Pgp expressed on the BBB behaves similarly in wild type and APPsw mice. ID, injected dose.

FIG. 4 depicts increased Aβ accumulation in APPsw, Pgp-null mice. (A and B) Brain sections from APPsw mice expressing or lacking Pgp were stained with an anti-Aβ antibody. Twelve month old APPsw, Pgp-null (mdr1a/b^{-/-}) mice showed greater Aβ deposition within the hippocampus and cortex then their Pgp-wild type littermates. Scale bar=250 μm. (C) As quantified by unbiased stereological techniques, Pgp-null mice exhibited a greater percentage of the hippocampus covered by Aβ immunoreactivity (% Aβ load) than Pgp wild type mice; **p=0.0041. The pattern of Aβ deposition within the hippocampus was similar in both genotypes. (D) In addition, the amount of fibrillar Aβ, as assessed by the percent area of hippocampus covered by thioflavine S staining (% thio load), was also increased in Pgp-null animals; *p=0.0276. (E) However, when normalized to Aβimmunoreactivity, the percentage of fibrillar Aβ to total Aβ deposits was not different between the groups; p=0.9711. (F) Fresh hippocampal tissue was sequentially extracted with carbonate, then guanidine and AβELISA was performed on the extracts. Aβ levels within each homogenate were normalized to the protein (prot.) content. Guanidine extracted, insoluble Aβ₄₂ was significantly elevated in Pgp-null mice, ^{#}p=0.0499, while there was only a trend for elevated insoluble Aβ₄₀, p=0.2452 (G). (H) Similar to the hippocampus, there was also a trend for greater Aβimmunoreactivity within the cortex of Pgp-null animals; p=0.2788. Values are mean±SEM, n=11-16 per group.

### DETAILED DESCRIPTION OF THE PREFERRED EMBODIMENTS

It has been discovered, as illustrated in the examples, that P-glycoprotein (Pgp; ABCB1) regulates Aβ levels in the brain. Pgp, the 170 kD protein product of the MDR1 gene, is an efflux transporter that belongs to the ATP binding-cassette (ABC) protein family. Pgp is highly expressed on the luminal surface of brain capillary endothelial cells wherein Pgp functionally comprises a major component of the BBB by limiting central nervous system penetration of various agents (Ambudkar, S. V., et al. 1999. Annu. Rev. Pharmacol. Toxicol. 39:361-398; Schinkel, A. H., et al. 1994. Cell. 77:491-502). In particular, it has been discovered that enhancement of Pgp function will decrease Aβ accumulation in the brain. Taken together, since increased Aβ levels in the brain contribute to Alzheimer's disease (AD) or other disorders mediated by amyloid-beta accumulation, the discoveries provide new treatment strategies for these diseases and disorders.

One aspect of the present invention encompasses a method to treat, prevent, or delay AD or other disorders mediated by amyloid-beta accumulation in a subject. The method may be utilized to treat a subject that is at risk of developing any of the AD indications described herein or to treat a subject that already has any of the indications described herein. Similarly, the method may be utilized to treat a subject that is at risk of developing any indication of another disorder mediated by amyloid-beta accumulation or to treat a subject that already has any of the indications described herein. Typically, the method comprises modulating the concentration of Aβ in the brain interstitial fluid. Another aspect of the present invention provides a method for modulating Aβ concentration in brain interstitial fluid comprised of modulating Pgp-mediated transport of Aβ across the BBB. Methods of measuring Aβ transport across the BBB are known in the art and outlined in the examples. Advantageously, Pgp is highly conserved across species. For example, in mice, Pgp is encoded by both mdr1a and mdr1b, which have 90% homology to each other and 80% to human MDR1 (Ambudkar, S. V., et al. 1999. Annu. Rev. Pharmacol. Toxicol. 39:361-398). In addition to mice and humans, a researcher in the field would appreciate that many other organisms express homologues of Pgp. In particular, Pgp homologues are expressed in non-human primates and in other rodents such as rats and hamsters. As such, the methods of the invention may be utilized in several subjects, including primates and rodents. In an exemplary embodiment, the subject is a human.

In one embodiment, the method comprises modulating the concentration of Aβ in the brain interstitial fluid of the subject. In a further embodiment, the method for treating AD or other disorders mediated by amyloid-beta accumulation in a subject comprises modulating the transport of Aβ from the brain interstitial fluid across the BBB of the subject. In still a further embodiment, Aβ transport across the BBB is modulated by altering the expression of Pgp at the BBB. For instance, in one embodiment, the transport of Aβ across the BBB is modulated by increasing the expression of Pgp at the BBB. Methods to increase Pgp expression are commonly known in the art. Some non-limiting examples include genetic engineering, upregulating Pgp transcription, upregulating Pgp translation, decreasing Pgp turnover, viral mediated gene therapy, somatic gene therapy, and germ-line gene therapy. Methods to detect increased expression of Pgp are also commonly known in the art, and include immunohistochemistry and Western blot analysis. In each of the above embodiments, the increased expression of Pgp at the BBB modulates the transport of Aβ across the BBB. Furthermore, in each of the above embodiments the increased expression of Pgp modulates the concentration of Aβ in brain interstitial fluid. Consequently, each of the above embodiments may be used to treat AD or other disorders mediated by amyloid-beta accumulation in a subject.

In an alternative embodiment, the transport of Aβ across the BBB is modulated by decreasing the expression of Pgp at the BBB. Methods to decrease Pgp expression are commonly known in the art. Some non-limiting examples include genetic engineering, downregulating Pgp transcription, downregulating Pgp translation, increasing Pgp turnover, RNA interference, viral mediated gene therapy, somatic gene therapy, and germ-line gene therapy. In one embodiment, Pgp expression is decreased by genetically engineering a cell that lacks at least one functional gene encoding Pgp. In another embodiment, Pgp expression is decreased by genetically engineering a cell that lacks two functional genes encoding Pgp. In yet another embodiment, Pgp expression is decreased by genetically engineering a cell that lacks more than two functional genes encoding Pgp. In another embodiment, Pgp expression is decreased by genetically engineering a mouse that lacks a functional Pgp protein. Methods to detect decreased expression of Pgp are commonly known in the art, and include immunohistochemistry and Western blot analysis. In each of the above embodiments, the decreased expression of Pgp at the BBB modulates the transport of Aβ across the BBB. Furthermore, in each of the above embodiments the decreased expression of Pgp modulates the concentration of Aβ in brain interstitial fluid.

Alternatively, the invention encompasses modulating the transport of Aβ across the BBB by modulating the expression of Pgp polymorphisms. In one embodiment, the transport of Aβ across the BBB increases with the expression of a Pgp polymorphism. In another embodiment, the transport of Aβ across the BBB decreases with the expression of a Pgp polymorphism. Non-limiting examples of Pgp polymorphisms are listed in Table 1. Methods to express a polymorphism of Pgp are known in the art and include genetic engineering, viral mediated gene therapy, somatic gene therapy, or germ-line gene therapy. In one embodiment, a Pgp polymorphism is expressed in rodents. In another embodiment, a Pgp polymorphism is expressed in mice. In yet another embodiment, a Pgp polymorphism is expressed in primates. In still yet another embodiment, a Pgp polymorphism is expressed in humans. Alternatively, in each of the above embodiments the expression of a Pgp polymorphism may be increased or decreased to affect the transport of Aβ. Methods to increase or decrease expression of a Pgp polymorphism are commonly known in the art. In each of the above embodiments, the induced, decreased, or increased expression of a Pgp polymorphism modulates the transport of Aβ. Furthermore, in each of the above embodiments the induced, decreased, or increased expression of a Pgp polymorphism modulates the concentration of Aβ in brain interstitial fluid. Consequently, each of the above embodiments may be used to treat AD or other disorders mediated by amyloid-beta accumulation.

Another aspect of the invention encompasses modulating the transport of Aβ from the brain interstitial fluid across the BBB by modulating the activity of Pgp. In one embodiment, the transport of Aβ across the blood brain barrier may be modulated by decreasing the activity of Pgp. Methods and compounds that decrease the activity of Pgp are known in the art. In one embodiment, a compound to decrease the activity of Pgp may be selected from the group comprising antihypertensive agents, antiadrenergics, calcium channel blockers, antiarrhythmics, immunosuppressive compounds, antibiotics, hormones, protease inhibitors, statins, antimycotics, antipsychotics, opioids, selective serotonin reuptake inhibitors, dopamine agonists, platelet aggregation inhibitors, and quinine derivatives. In one embodiment, the antihypertensive agents are selected from the group comprising antiadrenergics, calcium channel blockers, spironolactone, and reserpine. In another embodiment, the antiadrenergics are selected from the group comprising alpha blockers and beta blockers. In yet another embodiment, the antiadrenergics are selected from the group comprising carvedilol, atenolol, metaprolol, oxprenolol, pindolol, and propanolol. In a further embodiment the antiadrenergics is carvedilol. In one embodiment, calcium channel blockers are selected from the group comprising lercanidipine, nisolipine, dihydropyridine calcium channel blockers, phenylalkylamine calcium channel blockers, benxothiazepine calcium channel blockers, and menthol. In another embodiment, the calcium channel blockers are selected from the group comprising phenylalkylamine calcium channel blockers. In yet another embodiment, the calcium channel blocker is verapamil. Alternatively, the calcium channel blockers are selected from the group comprising dihydropyridine calcium channel blockers. In another alternative, the calcium channel blocker is nicardipine. In one embodiment, the antiarrhythmatics are selected from the group comprising class I, class II, class III, class IV, and class V antiarrhythmatics. In another embodiment, the antiarrhythmatics are selected from the group comprising class IA, class IB, and class IC antiarrhythmatics. In yet another embodiment, the antiarrhythmatic is propafenone or quinidine. Alternatively, the antiarrhythmatic is selected from the group comprising class III antiarrhythmatics. In another alternative, the antiarrhythmatic is amiodarone. In one embodiment, the immunosuppressive compounds may be selected from the group comprising glucocorticoids, cytostatics, antibodies, cyclosporine, tacrolimus, sirolimus, interferons, opioids, TNF-binding proteins, valspodar, and mycophenolate mofetil. In another embodiment, the immunosuppressive compound is selected from the group comprising drugs acting on immunophilins. In yet another embodiment, the immunosuppressive compound is cyclosporine A. In one embodiment, antibiotics may be selected from the group comprising aminoglycosides, beta-lactam ring antibiotics, glycopeptide antibiotics, oxazolidinones, polyketides, polymyxins, quinolones, fluoroquinolones, streptogramins, sulfonamides, chloramphenicol, clindamycin, fusidic acid and trimethoprim. In another embodiment, antibiotics may be selected from the group comprising polyketides. In yet another embodiment, antibiotics may be selected from the group comprising macrolides. In still another embodiment, the antibiotic is erythromycin or clarithromycin. In one embodiment, hormones may be selected from the group comprising amine-derived hormones, peptide hormones, steroid hormones, and lipid/phospholipid hormones. In another embodiment, hormones may be selected from the group comprising steroid hormones. In yet another embodiment, hormones may be selected from the group comprising sex steroids. In still another embodiment, the hormone is progesterone. In one embodiment, protease inhibitors are selected from the group comprising biological and pharmacological protease inhibitors. In another embodiment, protease inhibitors are selected from the group comprising HIV protease inhibitors. In yet another embodiment, protease inhibitors are selected from the group comprising indinavir, nelfinavir, ritonavir, and saquinavir. In one embodiment, the antipsychotics are selected from the group comprising typical antipsychotics, atypical antipsychotics, and partial dopamine agonists. In another example, the antipsychotics are selected from the group comprising typical antipsychotics. In yet another example, the antipsychotic is chloropromazine or flupenthixol. Non-limiting examples of statins include both fermentation-derived statins and synthetic statins. In one embodiment, fermentation-derived statins may be selected from the group comprised of lovastatin, simvastatin, and pravastatin. In another embodiment, synthetic statins may be selected from the group comprised of fluvastatin, atorvastatin, cerivastatin, and rosuvastatin. In yet another embodiment, the statin is atorvastatin. In one embodiment, the antimycotics are selected from the group comprising polyene antibiotics, imidazole antimycotics, triazole antimycotics, allylamine antimycotics, echinocandins, flucytosine, and griseofulvin. In another embodiment, the antimycotics are selected from the group comprising imidazole and triazole antimycotics. In yet another embodiment, the antimycotics are itraconazole or ketoconazole. In one embodiment, selective serotonin reuptake inhibitors may be selected from the group comprising fluoxetine, sertraline, escitalopram oxalate, citalopram, fluvoxamine maleate, and paroxetine. In another embodiment, the selective serotonin reuptake inhibitor is fluoxetine, sertraline, or paroxetine. In one embodiment, opioids are selected from the group comprising endogenous opioids, opium alkaloids, semisynthetic opium derivatives, synthetic opioids, phenylhetylamines, phenylpiperideines, diphenylpropylamine derivatives, benxomorphan derivatives, oripavine derivatives, morphinan derivates, dezocine, etorphine, tilidine, tramadol, loperamide, and diphenoxylate. In another embodiment, the opioid is selected from the group comprising phenylhepylamines and benzomorphan derivatives. In yet another embodiment, the opioid is methadone or pentazocine. In one embodiment the dopamine agonist is bromocriptine. In another embodiment, the platelet aggregation inhibitor is dipyridamole. In yet another embodiment, the quinine derivative is mefloquine. In each of the above embodiments, the decreased activity of Pgp modulates the transport of Aβ. Furthermore, in each of the above embodiments the decreased activity of Pgp modulates the concentration of Aβ in brain interstitial fluid.

In an alternative embodiment, the transport of Aβ across the blood brain barrier may be modulated by increasing the activity of Pgp. Methods and compounds that increase the activity of Pgp are known in the art. In one embodiment compounds to increase the activity of Pgp are selected from the group comprising hormones, opioids, antibiotics, flavonoids, antidepressants, protease inhibitors, antipsychotics, and retinoid derivatives. In a further embodiment, hormones are selected from the group comprising amine-derived hormones, peptide hormones, steroid hormones, and lipid/phospholipid hormones. In another embodiment, hormones may be selected from the group comprising steroid hormones. In yet another embodiment, hormones may be selected from the group comprising natural or synthetic glucocorticoids. In still another embodiment the hormone is dexamethasone. In one embodiment, opioids are selected from the group comprising endogenous opioids, opium alkaloids, semisynthetic opium derivatives, synthetic opioids, phenylheptylamines, phenylpiperideines, diphenylpropylamine derivatives, benxomorphan derivatives, oripavine derivatives, morphinan derivates, dezocine, etorphine, tilidine, tramadol, loperamide, and diphenoxylate. In another embodiment the opioid is selected from the group comprising opium alkaloids. In yet another embodiment, the opioid is morphine. In one embodiment, an antibiotic may be selected from the group comprising aminoglycosides, beta-lactam ring antibiotics, glycopeptide antibiotics, oxazolidinones, polyketides, polymyxins, quinolones, fluoroquinolones, streptogramins, sulfonamides, chloramphenicol, clindamycin, fusidic acid and trimethoprim. In another embodiment, antibiotics may be selected from the group comprising polyketides. In still another embodiment, antibiotics may be selected from the group comprising rifamycins. In yet still another embodiment, the antibiotic is rifampin. In one embodiment, the flavonoid is selected from the group comprising flavonols, flavones, flavanones, flavan-3-ols, isoflavones, and anthocyanidins. In another embodiment the flavonoid is selected from the group comprising flavonols. In still another embodiment, the flavonoid is quercetin. In one embodiment the antidepressant is selected from the group comprising serotonin reuptake inhibitors. In another embodiment, the antidepressant is selected from the group comprising hyperforin and hypericin. In yet another embodiment, the antidepressant is St. John's wort. In one embodiment, protease inhibitors are selected from the group comprising biological and pharmacological protease inhibitors. In another embodiment, protease inhibitors are selected from the group comprising HIV protease inhibitors. In yet another embodiment, protease inhibitors are selected from the group comprising amprenavir, indinavir, nelfinavir, ritonavir, and saquinavir. In one embodiment, the antipsychotics are selected from the group comprising typical antipsychotics, atypical antipsychotics, and partial dopamine agonists. In another example, the antipsychotics are selected from the typical antipsychotics. In yet another example, the antipsychotic is phenothiazine. In one embodiment, the retininoid derivative is retinoic acid. In each of the above embodiments, the increased activity of Pgp modulates the transport of Aβ. Furthermore, in each of the above embodiments the increased activity of Pgp modulates the concentration of Aβ in brain interstitial fluid. Consequently, each of the above embodiments may be used to treat AD or other disorders mediated by amyloid-beta accumulation.

Therapeutic agents, such as any of the compounds or compositions described above or otherwise known in the art, utilized in the present invention for treating AD or other disorders mediated by amyloid-beta accumulation may be in the form of free bases or pharmaceutically acceptable acid addition salts thereof. The term “pharmaceutically-acceptable salts” embraces salts commonly used to form alkali metal salts and to form addition salts of free acids or free bases. The nature of the salt may vary, provided that it is pharmaceutically acceptable. Suitable pharmaceutically acceptable acid addition salts of compounds of use in the present methods may be prepared from an inorganic acid or from an organic acid. Examples of such inorganic adds are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound.

As will be appreciated by the skilled artisan, the therapeutic agents of the present invention may be formulated into pharmaceutical compositions and administered by a number of different means that will deliver a therapeutically effective dose. They may be administered locally or systemically. Such compositions may be administered orally, parenterally, by inhalation spray, intrapulmonary, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intraarterial, intraperitoneal, intracochlear, or intrasternal injection, or infusion techniques. The therapeutic agents of the present invention may be administered by daily subcutaneous injection or by implants. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Those skilled in the art will appreciate that dosages and therapeutically effective amounts for treating AD or other disorders mediated by amyloid-beta accumulation in accordance with the methods of the invention may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

### DEFINITIONS

As used herein, Aβ refers to a fragment of amyloid precursor protein. Aβ is also referred to as beta-amyloid protein or amyloid beta protein.

As used herein, “agent” and “compound” are used interchangeably. Typically the agent or compound is administered to a subject to treat AD or other disorders mediated by amyloid-beta accumulation by modulating Pgp.

As used herein, the blood brain barrier (BBB) refers to the physical barrier between the lumen of brain capillaries and the brain itself formed in part by the capillary epithelial cells.

As used herein, Alzheimer's disease (AD) refers to dementia, disease, or disorder associated with the accumulation of Aβ in the parenchyma of the brain and/or in the cerebral arterioles in the form of cerebral amyloid angiopathy (CAA).

An “effective amount” is a therapeutically-effective amount that is intended to qualify the amount of an agent or compound, that when administered to a subject, will achieve the goal of preventing, delaying, or treating the cognitive loss associated with dementia due to AD or other disorders mediated by amyloid-beta accumulation.

The term “modulate,” as used herein, is used in its broadest interpretation and refers to a change in the biological activity of a biologically active molecule. Modulation may be an increase or a decrease in activity, a change in binding characteristics, or any other change in the biological, functional, or immunological properties of biologically active molecules. In an exemplary embodiment, “modulation of Pgp activity” refers to a change in Pgp mediated transport of Aβ across the BBB.

As used herein, polymorphism refers to variations in a genetic sequence present in a population. Polymorphisms may be single nucleotide changes in a genetic sequence, or they might involve more than a single nucleotide.

The term “treat” or “treatment” as used herein in the context of AD or other disorders mediated by beta-amyloid accumulation, includes preventing the damage before it occurs, or reducing loss or damage after it occurs.

### EXAMPLES

### Materials and Methods

### Animals:

All experimental procedures involving animals were performed in accordance with guidelines established by the Animal Studies Committee at Washington University. We utilized 8-10 week old mdr1a/b^{-/-} double-knockout mice on an FVB background (Taconic Farms) and wild type controls (also on an FVB background) for our BBB transport studies. We also bred mdr1a/b^{-/-} mice to APPsw^{+/-} hemizygous mice (Tg2576 on a C57BL/6-SJL background; a generous gift from Dr. K. Ashe, University of Minnesota). We then bred F₁ offspring from this colony to each other to produce littermate mdr1 a/b homozygous (+/+) and double-knockout (−/−) mice that were hemizygous for the APPsw^{+/-} transgene. Animals were screened for the presence of APPsw as well as mdr1a and mdr1b genes by PCR analysis of tail DNA. Aβ content in the brain was analyzed when animals were 12 months old.

### BBB Transport:

Transport of human [¹²⁵I]Aβ₄₀ or [¹²⁵I]Aβ₄₂ across the BBB and the associated calculations were performed as described (DeMattos, R. B., et al. 2002. Science. 295:2264-2267; Zlokovic, B. V., et al. 2000. Nat. Med. 6:718-719.). Briefly, a guide cannula was implanted stereotaxically into the right caudate-putamen of anesthetized mice at coordinates 0.9 mm anterior to bregma, 1.9 mm lateral, and 2.9 mm below the dura. Animals were allowed to recover after surgery prior to radiotracer studies. The clearance experiments were performed before substantial chronic processes occurred, typically 4-6 hours following cannula implantation, to allow the BBB to at least partially repair and exclude large molecules (8, 16). Twelve nanomoles of [¹⁴C]inulin and either [¹²⁵I]Aβ₄₀ or [¹²⁵I]Aβ₄₂ was co-injected in a volume of 0.5 μl over 5 minutes using an ultra micropump (World Precision Instruments). The specific activities of peptide were in the range of 80-120 μCi/μg. The percentage of radioactivity remaining in the brain after microinjection was determined as % recovery in brain=100×(N_{b}/Nᵢ), where N_{b} is the radioactivity remaining in the brain at the end of the experiment and Nᵢ is the radioactivity microinjected into brain ISF, i.e., the disintegrations per minute for [¹⁴C]inulin and the counts per minute for tricholoracetic acid-precipitable [¹²⁵I]radioactivity (intact Aβ). The percentage of Aβ cleared through the BBB was calculated as [(1−N_{b(Aβ)}/N_{i(Aβ)})−(1−N_{b(inulin)}/Nᵢ₍ᵢₙᵤₗᵢₙ₎)]×100, using a standard time of 30 min.

D-[^{4,5-3}H]leucine (Amersham Biosciences) transport across the BBB was assessed using a method similar to the that described above; 750 nanomoles of [³H]leucine, specific activity 1.16 Ci/mg, was injected in a volume of 0.5 μl followed by measurement of radioactivity within the brain at 10 minutes.

### ISF Aβ In Vivo Microdialysis:

In vivo microdialysis to assess brain ISF Aβ₁₋ₓ in the hippocampus of awake, freely moving 3 month old APPsw^{+/-} mice was performed exactly as described (Cirrito, J. R., et al. 2003. J. Neurosci. 23:8844-8853; DeMattos, R. B., et al. 2004. Neuron. 41:193-202). Aβ₁₋ₓ represents all Aβ peptides beginning at amino acid 1 of the N-terminus with the C-terminus being variable but predominantly either ending at position 40 or 42. An initial 4-6 hour recovery period elapsed after guide implantation and probe insertion to allow for tissue recovery, followed by collection of microdialysis samples for 5 hours at one-hour intervals at a flow rate of 1.5 μl/min to establish basal level of ISF Aβ. XR9576 (80 mg/kg body weight; Xenova QLT Inc.; diluted in 5% dextrose), or vehicle was injected into the jugular vein and an additional 10 one-hour microdialysis samples were collected. Concentrations of ISF Aβ₁₋ₓ for each mouse were expressed as the percentage of basal level for each mouse (mean of 5 hours prior to treatment).

[(^{99m}Tc]Sestamibi Transport and Biodistribution.

The radiopharmaceutical [^{99m}Tc]Sestamibi (Bristol-Myers Squibb Co.) was prepared from a one-step commercial kit formulation by addition of [^{99m}Tc]TcO₄⁻ according to the manufacturer's recommendations (25). Sep-Pak purification (>95%) and quality control of the tracer were performed as previously described (Piwnica-Worms, D., et al. 1995. Biochemistry. 34:12210-12220). Following intravenous injection, distribution of [^{99m}Tc]Sestamibi in the brain and blood of Pgp-wild type and Pgp-null mice with or without co-expression of APPsw^{+/-} was determined 5 minutes post-injection as previously described (Chen, W. S., et al. 2000. Biochem. Pharmacol. 60:413-426; Luker, G. D., et al. 1997. Biochemistry. 36:14218-14227). Data are expressed as percent injected dose per gram tissue [(μCi [^{99m}Tc]Sestamibi in tissue) (μCi injected [^{99m}Tc]Sestamibi)⁻¹(g tissue)⁻¹×100] and are reported as mean±SEM (n=3-4) or ±range (n=2).

### Histological Analysis.

Tissue sections were cut in the coronal plane at 50 μm on a freezing sliding microtome from the genu of the corpus callosum through the caudal extent of the hippocampus. The percent surface area covered by Aβ-immunoreactivity deposits (% Aβload), as identified with a mouse monoclonal antibody against the N-terminus of human Aβ, m3D6 (Eli Lilly and Co.), was quantified following unbiased stereological principles as described (Holtzman, D. M., et al. 2000. Ann. Neurol. 47:739-747.). Thioflavine-S staining was performed stereologically and quantified as previously described (Bales, K. R., et al. 1997. Nat. Genet. 17:263-264.). Aβ and thioflavine-S load were determined in the cingulate cortex and hippocampus in three brain sections, each separated by 300 μm.

### Aβ Quantification.

Aβ₄₀ and Aβ₄₂ levels in brain tissue were determined by sandwich ELISA as previously described (Cirrito, J. R., et al. 2003. J. Neurosci. 23:8844-8853.). Briefly, m266, m2G3, or m21F12 were used as coating antibodies to capture Aβ₁₋ₓ, Aβ₄₀, or Aβ₄₂, respectively. Biotinylated m3D6, a human-specific antibody against the N-terminus of Aβ, was used as the reporting antibody in each ELISA. To evaluate the carbonate soluble and insoluble pools of Aβ in brain tissue, we performed a carbonate extraction (100 mM carbonate, 50 mM NaCl, protease inhibitors, pH 11.5) of hippocampal tissue (1:10, wt:vol) on ice followed by a 5 M guanidine, pH 8.0 extraction for 3 hours at room temp. Tissue samples were dounce homogenized and spun in a microcentrifuge at 21,000 g for 15 minutes at 4° C. following each extraction.

### Western Blotting.

Hippocampal tissue from 12 month old APPsw, Pgp-wild type and APPsw, Pgp-null littermates was homogenized in 150 mM NaCl, 50 mM Tris pH 7.4, 0.5% deoxycholic acid, 0.1% SDS, 1% Triton X-100, 2.5 mM EDTA, and protease inhibitors. Western blotting for MRP1, LRP1, and RAGE were performed using 3-8% NuPAGE Tris-Acetate gels (Invitrogen) with thirty micrograms of protein loaded per lane. Cerebral microvessels from 2-3 month old PGP-wildtype (FVB wild type) and Pgp-null mice were isolated as described (Zlokovic, B. V., et al. 1993. J. Biol. Chem. 268:8019-8025.). Microvessels were homogenized in cold lysis buffer: 150 mM NaCl, 50 mM Tris pH 7.4, 0.1% SDS, 1% Triton X-100 and complete protease inhibitor cocktail (Roche). Individual protein homogenates were separated under non-reducing conditions, transferred to nitrocellulose membrane and probed with rabbit-anti-LRP1-85 (LRP, β-chain specific, EMD Biosciences Inc.), rabbit-anti-RAGE (Santa Cruz Biotechnology), or rat-anti-MRP1 (Signet Laboratories), followed by goat-anti-rabbit or donkey-anti-rat secondary antibodies conjugated to HRP (Jackson Immunoresearch Laboratories). Mouse-anti-tubulin (Sigma-Aldrich) or anti-β-actin (Santa Cruz Biotechnology) were used to detect control proteins in each lane. Bands were detected with Lumigen-TMA6 (Amersham Biosciences) and captured digitally using the Kodak ImageStation 440CF. The relative absorbance of each band was detected by densitometry and normalized to protein loaded in each lane. Analysis was performed using the Kodak 1 D Image Analysis software version 3.6.

### Statistical Analysis.

Data shown in figures represent mean±SEM. Mann-Whitney U test was used to compare data between Pgp-null and wild type mice, as well as LRP1 expression levels in endothelial cells of XR9576 and vehicle treated mice. ISF Aβ levels in XR9576 and vehicle treated mice, over the duration of the experiment, were compared by two-way ANOVA followed by the Bonferroni post-hoc test to compare means. P-values of less than 0.05 were considered statistically significant. All statistical analyses were performed using Prism version 4.02 (GraphPad software) for Windows (Microsoft).

### Example 1

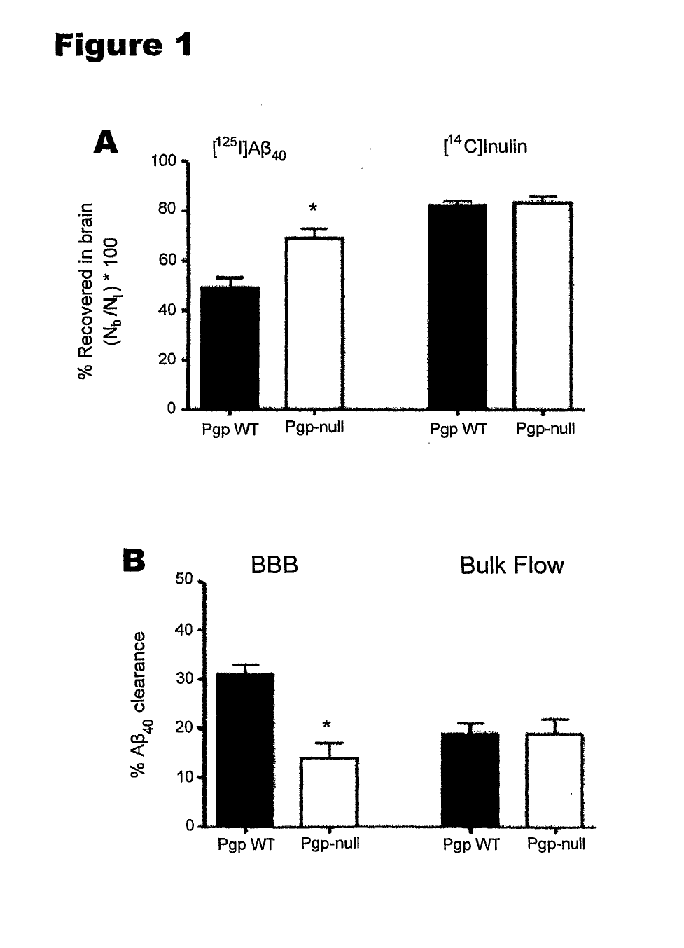

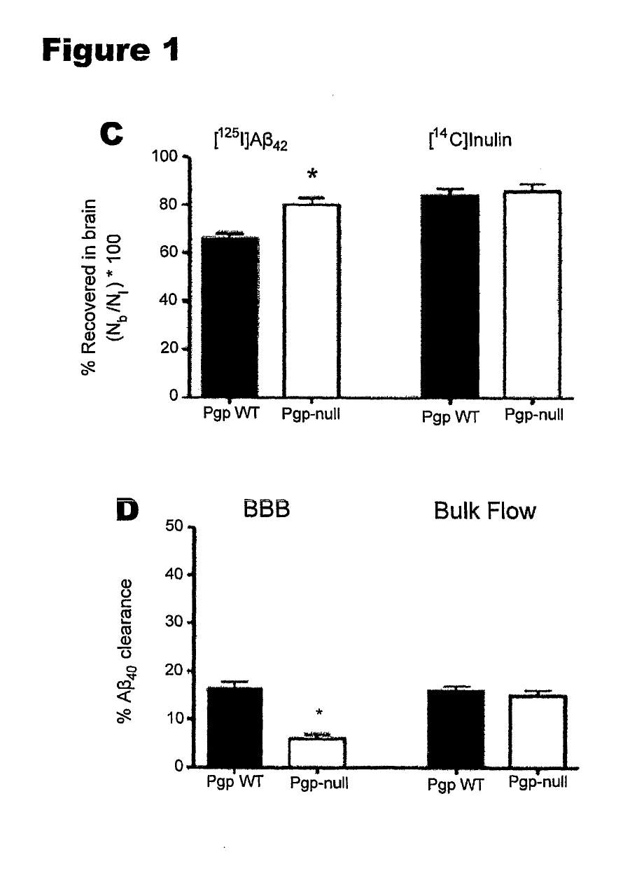

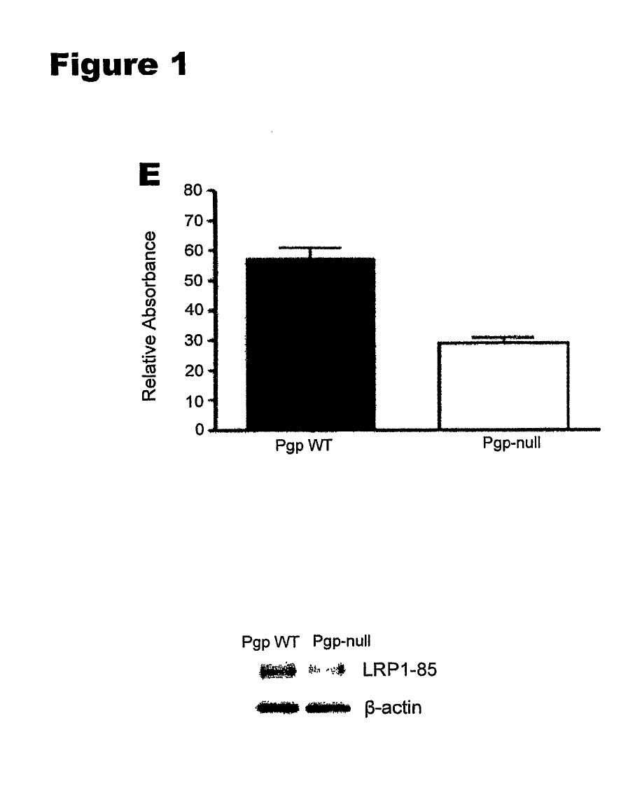

To directly assess the role of Pgp in transport of Aβ across the BBB, [¹²⁵I]Aβ₄₀ or [¹²⁵I]Aβ₄₂ was microinjected into the brain of mdr1a/b^{-/-} double-knockout (Pgp-null) mice and wild type controls (Deane, R., et al. 2004. Neuron. 43:333-344). [¹⁴C]inulin, a reference molecule that is neither actively transported across the BBB or retained within the brain, was co-administered to measure bulk flow of ISF in the same mice. Thirty minutes following co-administration of tracers, mice were sacrificed and the content of each compound remaining in the brain was assessed (FIGS. 1A and C). Significantly more [¹²⁵I]Aβ₄₀ and [¹²⁵I]Aβ₄₂ were retained within the brain of Pgp-null animals compared to Pgp-wild type mice (p=0.0123 and p=0.0047, respectively), strongly suggesting that Aβ elimination was hindered in these animals. Removal of [³H]leucine, a Pgp-independent, actively transported molecule, was unaffected at 10 minutes post-injection (61.4±3.8% and 62.7±5% retained in FVB WT versus Pgp-null, respectively). Similarly, bulk flow pathways, accounting for <20% of clearance, appeared unchanged in the knockout animals as demonstrated by nearly identical levels of inulin remaining in both groups. As shown in FIG. 1B, after passive elimination was taken into account, 31% of injected Aβ₄₀ was transported across the BBB in wild type mice whereas only ˜14% was cleared in the Pgp-null animals (p=0.0033). Similarly, 16% of Aβ₄₂ was transported in Pgp WT mice compared with 6% in Pgp-null mice (FIG. 1D). Compared with Aβ₄₀, significantly less Aβ₄₂ was removed from the brain in WT and Pgp-null mice which is consistent with previous studies showing that Aβ₄₀ is generally actively transported across the BBB more than Aβ₄₂ (Deane, R., et al. 2004. 43:333-344). LRP1 has been previously shown to transport Aβ across the BBB (Shibata, M., et al. 2000. J. Clin. Invest. 106:1489-1499; Deane, R., et al. 2004. Neuron. 43:333-344). While overall levels of LRP1 in brain homogenates were comparable (data not shown), interestingly, levels of LRP1 decreased by 50% in brain capillaries of 2-3 month old Pgp-null mice as compared to Pgp-wild type mice (FIG. 1E). This decrease in LRP1 expression at the BBB may have partially contributed to decreased Aβclearance in Pgp-null mice.

### Example 2

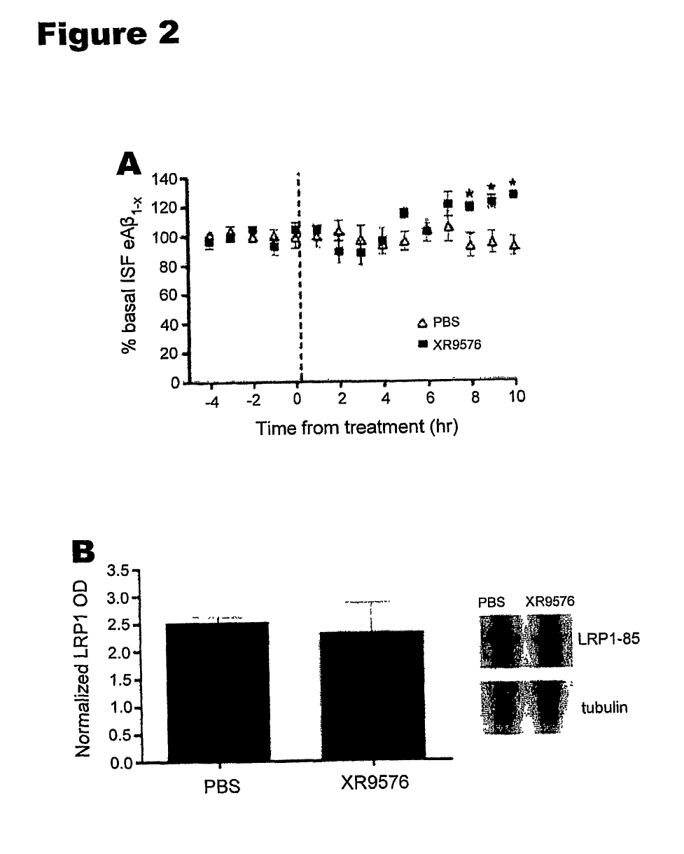

Acute inhibition of Pgp activity was assessed using APPsw mice (also called Tg2576). APPsw mice overexpress human amyloid precursor protein (APP) with a mutation that in humans causes an autosomal dominant form of early onset familial AD (Hsiao, K., et al. 1996. Science. 274:99-102.). A selective Pgp inhibitor, XR9576 (Mistry, P., et al. 2001. Cancer Res. 61:749-758.), was used to block Pgp activity at the BBB in APPsw mice. Because brain capillary endothelial cells transport molecules out of the brain ISF into the periphery, this fluid was directly assessed for changes in Aβ levels following intravenous administration of XR9576 at 80 mg/kg, a dose that inhibits Pgp function at the BBB as determined with [^{99m}Tc]Sestamibi analysis (data not shown). Using in vivo microdialysis in awake, freely moving mice (Cirrito, J. R., et al. 2003. J. Neurosci. 23:8844-8853; DeMattos, R. B., et al. 2004. Neuron. 41:193-202), a basal concentration of ISF Aβ was determined over 5 hours. Mice were then treated with either vehicle or XR9576 and ISF Aβ levels were assessed for an additional 10 hours (FIG. 2A). Eight hours after treatment, ISF Aβ levels increased almost 30% compared to vehicle treated mice (p<0.05), suggesting that acute inhibition of Pgp at the BBB caused decreased elimination of Aβ from brain ISF. Ten hours after treatment with either vehicle or XR9576, cerebral microvessels were also isolated and assessed for levels of LRP1 by Western blot. LRP1 levels in endothelial cells did not change (FIG. 2B), indicating that, in this case, the change in ISF Aβ levels is not attributable to LRP1 down-regulation, but likely due to specific inhibition of Pgp.

### Example 3

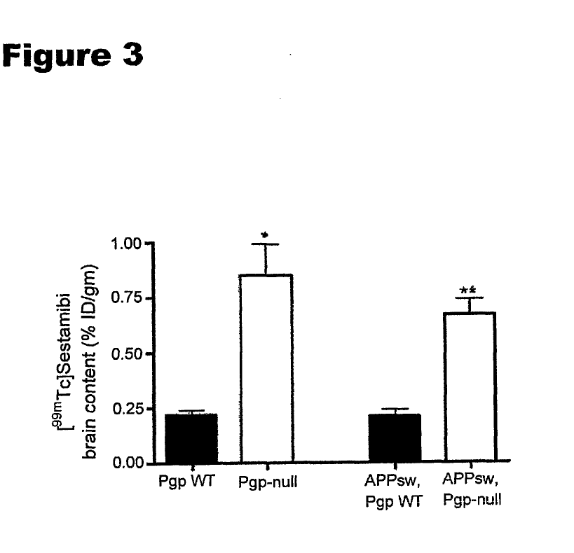

APPsw^{+/-} mice were bred to mdr1a/b^{-/-} animals to produce littermate mice that were positive for the APPsw transgene and either Pgp-null (APPsw^{+/-}, mdr1a/b^{-/-}) or Pgp wild type (APPsw^{+/-}, mdr1a/b^{+/+}). To confirm that transgenic expression of mutated human APP did not impact basal function of Pgp in vivo, biodistribution studies utilizing [^{99m}Tc]Sestamibi, a radiopharmaceutical that has been validated as a sensitive probe of Pgp transport function (Sharma, V., and Piwnica-Worms, D. 2005. Topics Curr. Chem. 252:155-178) were performed in 2-4 month old wild type (mdr1a/b^{+/+}) and Pgp-null (mdr1a/b^{-/-}) mice both in the absence and presence of the APPsw+/− transgene. Normally, [^{99m}Tc]Sestamibi is readily transported out of the brain by Pgp. However, in Pgp-null animals, [^{99m}Tc]Sestamibi levels increase in the brain as it permeates cerebral capillaries and is not removed effectively. Five minutes after intravenous bolus injection of [^{99m}Tc]Sestamibi (2 μCi), brain content of the tracer was 0.22±0.02% injected dose (ID)/gram (g) in wild type controls versus 0.85±0.14% ID/g in Pgp-null mice (FIG. 3). There was no difference in blood content of the tracer between the two strains (p>0.2). This approximately 3.5-fold increase in brain penetration of [^{99m}Tc]Sestamibi in Pgp-null mice was consistent with previous studies (Dyszlewski, M., et al. 2002. Mol. Imaging. 1:24-35). In similarly aged animals that expressed the APPsw+/− transgene, brain penetration of tracer was also increased by 3.5-fold in Pgp-null animals compared with those expressing Pgp (FIG. 3). Thus, basal activity of Pgp was independent of the APPsw+/− transgene (p>0.3).

### Example 4

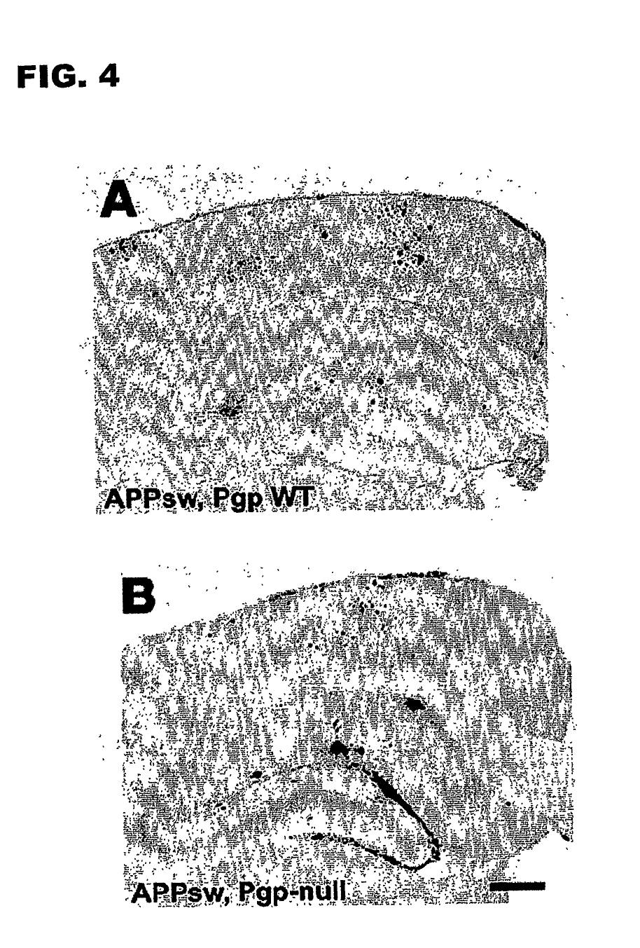

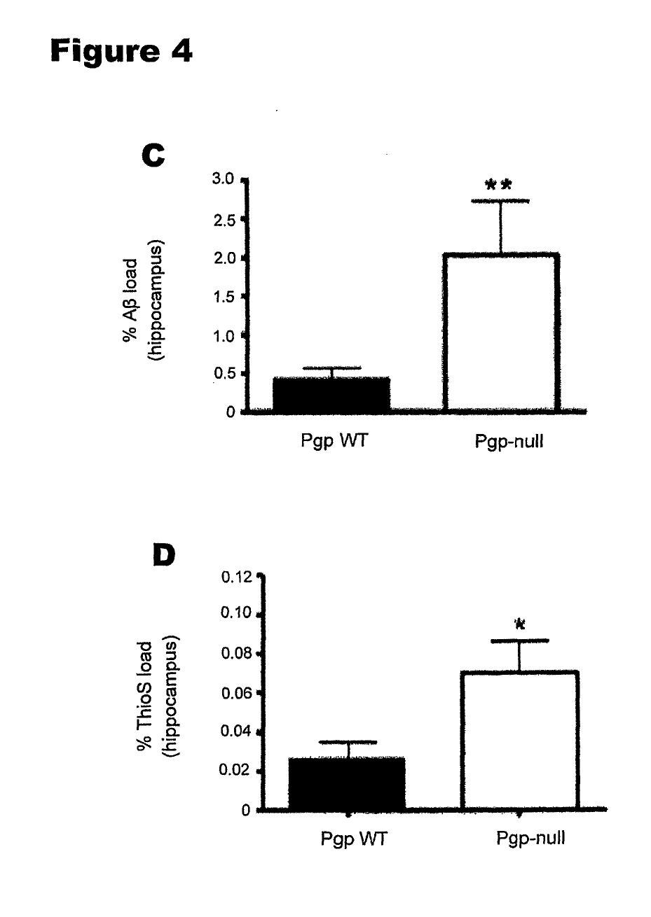

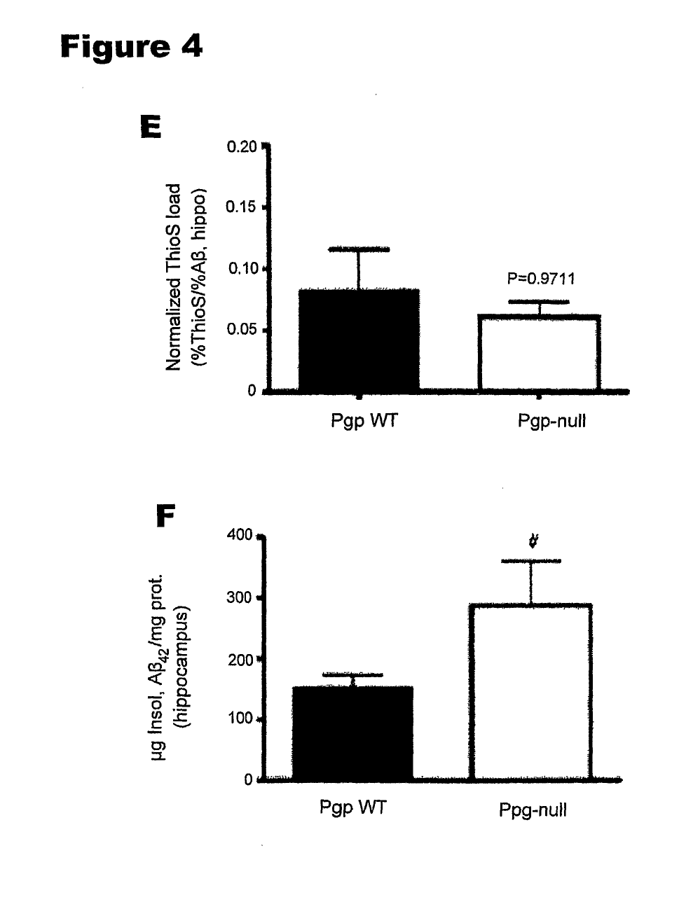

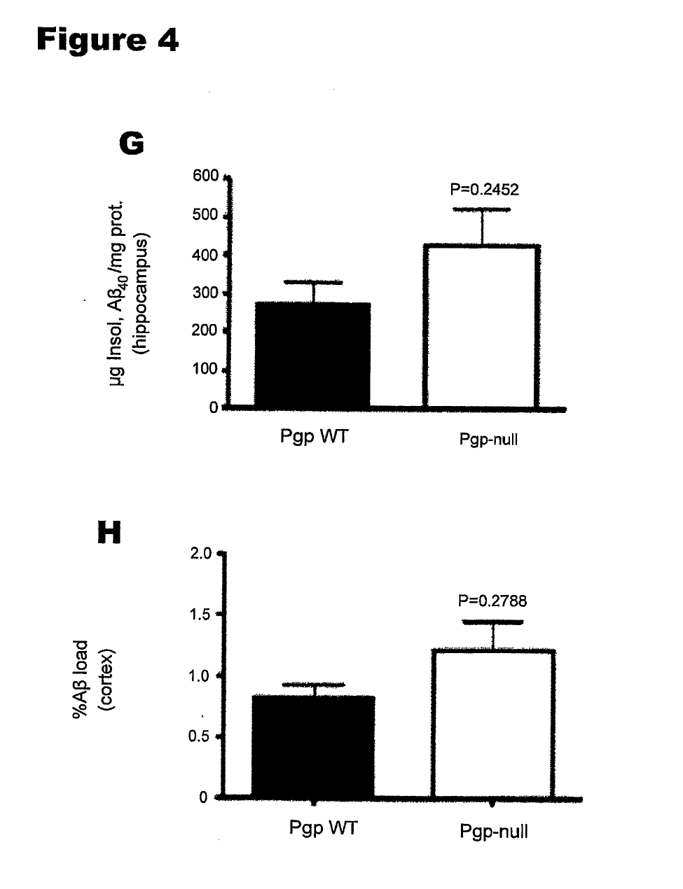

When allowed to age to 12 months, APPsw+/− mice exhibited substantial Aβ deposition throughout the neocortex and hippocampus (FIGS. 4A and B). The area covered by Aβ-immunoreactivity in the hippocampus was significantly greater in APPsw, Pgp-null animals compared with APPsw, Pgp-wild type littermates, p=0.0041 (FIG. 4C). The amount of fibrillar Aβ, as determined by thioflavine S staining, was also significantly elevated in Pgp-null animals, p=0.0276 (FIG. 4D). However, when the amount of thioflavine S was normalized to total plaque load, there was not a significant difference in the percentage of fibrillar plaques between groups (FIG. 4E). This suggests that while Pgp deletion leads to greater Aβ deposition, Pgp does not preferentially alter the conversion of plaques into fibriliar structures. There was no apparent change in the amount of vascular Aβ accumulation in 12-month old Pgp-null animals, however, it remains possible that at later ages, when there is a greater amount of cerebral amyloid angiopathy (CM), there may be a noticeable affect. The mass of insoluble Aβ₄₂, as assessed by guanidine extraction and AβELISA, was two-fold greater in the hippocampus of Pgp-null animals, p=0.0499 (FIG. 4F), while there was only a trend toward an increase in insoluble Aβ₄₀, p=0.2472 (FIG. 4G). Carbonate extractable Aβ₄₀ and Aβ₄₂ was not different between these groups (data not shown). Similar to the hippocampus, there was a trend toward elevated Aβ plaque load in the neocortex of Pgp-null mice, however this increase was not statistically significant (FIG. 4H).

These findings indicate that abrogation of the Pgp transporter at the BBB gave rise to greater Aβ accumulation within the brain and suggest that modulation of Pgp activity can directly influence progression of Aβ pathology. Because genetic deletion of Pgp also causes a decrease in the level of LRP1 at the BBB (FIG. 1), it remains possible that the changes in Aβ deposition are due to a combined decrease in both Aβ transporters. In fact, these Aβ transporters may be playing a synergistic role with LRP1 functional on the basolateral surface and Pgp on the luminal surface of brain endothelial cells.

Pgp shares sequence homology and substantial pharmacological cross-reactivity with several members of the ATP-binding cassette superfamily of transporters that are also expressed at the BBB, including MDR-associated protein 1 (MRP1; ABCC1) (Gottesman, M. M., Fojo, T., and Bates, S. E. 2002. Nat. Rev. Cancer. 2:48-58). In 12 month old APPsw, Pgp-null (APPsw^{+/-}, mdr1a/b^{-/-}) mice, expression of MRP1 in hippocampal homogenates was elevated compared to Pgp-wild type (APPsw^{+/-}, mdr1a/b^{+/+}) mice, p=0.0401 (data not shown). Although Pgp and MRP1 have overlapping pharmacological profiles, it is unknown whether MRP1 can also transport Aβ in vivo. In vitro studies suggest that MRP1 cannot transport Aβ (Lam, F. C., et al. 2001. J. Neurochem. 76:1121-1128.). RAGE, a transporter on the BBB that is responsible for transporting Aβ from the blood into the brain (Deane, R., et al. 2003. Nat. Med. 9:907-913; Deane, R., et al. 2004. Stroke. 35:2628-2631.), was expressed at similar levels in hippocampal homogenates of 12 month old APPsw, Pgp-null and APPsw, Pgp-wild type mice (data not shown).

TABLE 1

| Region | Position | Mutation |
|---|---|---|
| Promoter | 5′ flanking/−41 | A/G |
| Exon 1a | Exon 1a/−145 | C/G |
| Exon 1b | Exon 1b/−129 | T/C |
| Intron 1 | Exon 2/−4 | C/T |
| Intron 1 | Exon 2/−1 | G/A |
| Exon 2 | Exon 2/61 | A/G |
| Intron 4 | Exon 5/−35 | G/C |
| Intron 4 | Exon 5/−25 | G/T |
| Exon 5 | Exon 5/307 | T/C |
| Intron 6 | Exon 6/+139 | C/T |
| Intron 6 | Exon 6/+145 | C/T |
| Exon 7 | Exon 7/548 | A/G |
| Exon 11 | Exon 11/1199 | G/A |
| Exon 12 | Exon 12/1236 | C/T |
| Intron 12 | Exon 12/+44 | C/T |
| Exon 13 | Exon 13/1474 | C/T |
| Intron 16 | Exon 17/−76 | T/A |
| Intron 17 | Exon 17/+137 | A/G |
| Exon 21 | Exon 21/2650 | C/T |
| Exon 21 | Exon 21/2677 | G/T |
| Exon 24 | Exon 24/2956 | A/G |
| Exon 24 | Exon 24/2995 | G/A |
| Exon 26 | Exon 26/3320 | A/C |
| Exon 26 | Exon 26/3396 | C/T |
| Exon 26 | Exon 26/3421 | T/A |
| Exon 26 | Exon 26/3435 | C/T |
| Exon 28 | Exon 28/4030 | G/C |
| Exon 28 | Exon 28/4036 | A/G |

The positions of the polymorphisms were established with the first base of the ATG start codon set to 1. Mutations located in introns are given as position downstream (−) or upstream (+) of the respective exon. Modified from Marzolini, C., et al. 2004. Clin. Pharmacol. Ther. 75: 13-33.

What is claimed is:

1. A method of delaying the onset, slowing the progression, preventing or treating Alzheimer's disease in a subject, the method comprising modulating P-glycoprotein mediated transport of Aβ across the blood brain barrier of the subject.

2. The method of claim 1, wherein the transport of Aβ is modulated in the subject by increasing the expression of P-glycoprotein at the blood brain barrier.

3. The method of claim 1, wherein the transport of Aβ is modulated in the subject by inducing, increasing, or decreasing the expression of a P-glycoprotein polymorphism at the blood brain barrier.

4. The method of claim 1, wherein the transport of Aβ is modulated in the subject by increasing the activity of P-glycoprotein at the blood brain barrier.

5. The method of claim 4, wherein the activity of P-glycoprotein is increased by administering a compound to the subject selected from the group consisting of hormones, opioids, antibiotics, flavonoids, antidepressants, protease inhibitors, antipsychotics, and retinoid derivatives.

6. A method of modulating the concentration of Aβ in brain interstitial fluid of a subject, the method comprising modulating the P-glycoprotein mediated transport of Aβ across the blood brain barrier.

7. The method of claim 6, wherein the transport of Aβ is modulated in the subject by increasing or decreasing the expression of P-glycoprotein at the blood brain barrier.

8. The method of claim 6, wherein the transport of Aβ is modulated in the subject by inducing, increasing, or decreasing the expression of a P-glycoprotein polymorphism at the blood brain barrier.

9. The method of claim 6, wherein the transport of Aβ is modulated in the subject by increasing the activity of P-glycoprotein at the blood brain barrier.

10. The method of claim 9, wherein the activity of P-glycoprotein is increased by administering a compound to the subject selected from the group consisting of hormones, opioids, antibiotics, flavonoids, antidepressants, protease inhibitors, antipsychotics, and retinoid derivatives.

11. The method of claim 6, wherein the transport of Aβ is modulated in the subject by decreasing the activity of P-glycoprotein at the blood brain barrier.

12. The method of claim 11, wherein the activity of P-glycoprotein is decreased by administering a compound to the subject selected from the group consisting of antihypertensive agents, antiadrenergics, calcium channel blockers, antiarrhythmics, immunosuppressive compounds, antibiotics, hormones, protease inhibitors, statins, antimycotics, antipsychotics, opioids, selective serotonin reuptake inhibitors, dopamine agonists, platelet aggregation inhibitors, and quinine derivatives.

13. A method to modulate transport of Aβ from the brain interstitial fluid across the blood brain barrier in a subject, the method comprising modulating the activity of P-glycoprotein.

14. The method of claim 13, wherein the activity of P-glycoprotein is modulated in the subject by increasing or decreasing the expression of P-glycoprotein at the blood brain barrier.

15. The method of claim 13, wherein the activity of P-glycoprotein is modulated in the subject by inducing, increasing, or decreasing the expression of a P-glycoprotein polymorphism at the blood brain barrier.

16. The method of claim 13, wherein the transport of Aβ is modulated in the subject by increasing the activity of P-glycoprotein at the blood brain barrier.

17. The method of claim 16, wherein the activity of P-glycoprotein is increased by administering a compound to the subject selected from the group consisting of hormones, opioids, antibiotics, flavonoids, antidepressants, protease inhibitors, antipsychotics, and retinoid derivatives.

18. The method of claim 13, wherein the transport of Aβ is modulated in the subject by decreasing the activity of P-glycoprotein at the blood brain barrier.

19. The method of claim 18, wherein the activity of P-glycoprotein is decreased by administering a compound to the subject selected from the group comprising antihypertensive agents, antiadrenergics, calcium channel blockers, antiarrhythmics, immunosuppressive compounds, antibiotics, hormones, protease inhibitors, statins, antimycotics, antipsychotics, opioids, selective serotonin reuptake inhibitors, dopamine agonists, platelet aggregation inhibitors, and quinine derivatives.
